(19) United States
(12) Patent Application Publication
(10) Pub. No.: US 2014/0364866 A1
(43) Pub. Date: Dec. 11, 2014

(54) ENDOSCOPIC SNARE DEVICE

(71) Applicant: University of Louisville Research Foundation, Inc., Louisville, KY (US)

(72) Inventors: Gerald W. Dryden, Louisville, KY (US); Joseph Watson Vicars, III, Louisville, KY (US); Guruprasad A. Giridharan, Louisville, KY (US); Lauren Elizabeth Allen, Dublin, CA (US); Megan C. Mann, Louisville, KY (US); John F. Naber, Goshen, KY (US)

(73) Assignee: UNIVERSITY OF LOUISVILLE RESEARCH FOUNDATION, INC., Louisville, KY (US)

(21) Appl. No.: 14/370,617

(22) PCT Filed: Jan. 7, 2013
(86) PCT No.: PCT/US2013/020457

§ 371 Date: Jul. 3, 2014

Related U.S. Application Data
(60) Provisional application No. 61/583,785, filed on Jan. 6, 2012.
(60) Provisional application No. 61/648,312, filed on May 17, 2012.

(51) Int. Cl. A61B 17/221

(52) U.S. Cl. CPC A61B 17/221; A61B 2017/2212
USPC 606/113

(57) ABSTRACT

Embodiments of this invention relate to endoscopic and laparoscopic surgical instruments. More specifically, embodiments of this invention relate to an open loop snare device including a means for securely closing the loop. In some embodiments, a snare device comprises a snare wire and a capture mechanism wherein, when the snare wire is advanced, the snare wire extends from a distal portion of the device along an arcuate path curving back toward the capture mechanism such that, after the snare wire is advanced, activation of the capture mechanism captures the snare wire, creating a formed loop around a target tissue. Retraction of at least one of the snare wire and capture mechanism contracts the formed loop, resecting the target tissue.

This application claims the benefit of U.S. provisional patent application Ser. No. 61/648,312, filed May 17, 2012, for ENDOSCOPIC SNARE DEVICE, incorporated herein by reference. This application also claims the benefit of U.S. provisional patent application Ser. No. 61/583,785, filed Jan. 6, 2012, for POLYPECTOMY SNARE DEVICE, incorporated herein by reference.

### FIELD OF THE INVENTION

Embodiments of this invention relate to endoscopic and laparoscopic surgical instruments. More specifically, embodiments of this invention relate to an open loop snare device including a means for securely closing the loop. In some embodiments, a snare device comprises a snare wire and a capture mechanism wherein, when the snare wire is advanced, the snare wire extends from a distal portion of the device along an arcuate path curving back toward the capture mechanism such that, after the snare wire is advanced, activation of the capture mechanism captures the snare wire, creating a formed loop around a target tissue. Refraction of at least one of the snare wire and capture mechanism contracts the formed loop, resecting the target tissue.

### BACKGROUND

Polyps are routinely found in the mucosal lining of colons and rectums of adults over the age of 50 years. In addition to the colon and rectum, polyps can develop in the mucosal lining of other hollow organs and cavities in the body, including the small intestine, ear, nose, sinuses, stomach, and uterus. Removal of colon polyps has been strongly associated with a reduction in the incidence of colorectal cancer.

Polyps can be removed by surgical and endoscopic methods. Many polyps are routinely removed by biopsy forceps or closed loop endoscopic snares followed by cauterization or the application of a ligature. Certain types of polyps can be difficult to remove. Difficult polyps include large pedunculated polyps with thick pedicles and/or large heads as well as sessile polyps that have little to no stalk. Polyps may also be difficult to remove due to their location or multiple polyps clustering together. These difficult polyps present challenges to the clinician using existing snare technology.

The endoscopic treatment of difficult polyps presents unique challenges to the safety and efficacy of polypectomies. Encircling a large polyp is often difficult with existing closed loop snares as the size of the polyp inhibits the seating of the snare's loop around the base of the polyp which in turn prevents the typically necessary step of cauterization used to facilitate removal. Sessile polyps (i.e. large, flat polyps) often require the creation of a saline pillow beneath the polyp by using a submucosal saline injection technique to separate the tissue from the colon wall to reduce the chance of removal resulting in thermal injury or perforation of the colon wall.

Snare polypectomy is made more difficult when polyps are located in the right colon and cecum, behind folds, flexures and turns, or when polyps present themselves in an arrangement which creates access complications relative to the path of the endoscopic snare. Proper alignment can be particularly difficult to obtain when resecting a polyp from the medial wall of the cecum, just proximal to the ileocecal valve or at a flexure, or bend, of the colon. Polyp resection in the cecum and ascending colon are especially problematic due to the thinness of the colon wall which increases the risk of perforation or transmural burn.

Clinicians have dealt with complicated polypectomies in a number of ways. Difficult polyps are often removed piecemeal, sometimes over several procedures. The removal of colorectal polyps in this manner increases the risk to the patient, adds to the backlog of procedures scheduled for surgical suites, and unnecessarily consumes the valuable time of the clinician and support staff. Large sessile polyps located in sharp sigmoid bends can also be difficult and require individualized approaches, especially when utilizing conventional closed loop polypectomy snares.

Snare polypectomy with cutting electrosurgical currents is a well-regarded currently available method for complete polyp removal. Polypectomy snares are monofilament, braided or coiled wire snares shaped into a closed loop at the end of an endoscopic tool. Snares are typically categorized as mini (<11 mm diameter) or standard (15 mm-45 mm diameter). Snare geometries also vary widely and closed loop snares include oval, hexagonal, crescent, and circular-shaped loops.

### SUMMARY

The disclosed snare device is an open loop snare device with a capture mechanism to secure an extended snare wire, thereby forming a loop. The diameter of the formed loop may then be decreased, resecting tissue surrounded by the formed loop. The loop used to resect tissue is controllably formed by the user at a desired location in contrast to existing closed loop snares, wherein closed loops must be maneuvered over and around obstacles to reach the desired location.

In some embodiments, the disclosed snare device is also capable of delivering an electrosurgical current for the purpose of incision and/or to control bleeding by tissue desiccation or cauterization. In a further embodiment, the snare is also capable of rotation to facilitate arrangement within the colon with respect to the location of a target polyp. A shape memory wire is preferably utilized for the snare wire in some embodiments so as to facilitate the return of the shape memory wire along a predetermined arcuate path for capture. The shape memory wire is constructed from a shape memory material such as a shape memory alloy or a shape memory polymer. In some embodiments, the capture mechanism is a pair of opposing jaws capable of transitioning from an open to a closed position, thereby capturing the snare wire between them in a capture orifice. In other embodiments, the capture mechanism is a closed loop capable of being retracted to decrease its diameter, thereby capturing the snare wire after it has passed through the closed loop. Further embodiments entail using a conventional magnet or an electro-magnet to help guide and/or secure the distal end of the open looped endoscopic snare as it advances around the polyp and arcs back toward the distal end of the snare device.

In some embodiments, a snare device comprises a snare wire and a capture mechanism. When the snare wire is advanced, the snare wire extends from a distal portion of the device along an arcuate path curving back toward the capture mechanism. After the snare wire is advanced at least to the capture mechanism, activation of the capture mechanism captures the snare wire, creating a formed loop. Retraction of at least one of the snare wire and capture mechanism contracts the formed loop. In certain embodiments, the capture mechanism is a closed loop capable of capturing the snare wire by retracting, decreasing the diameter of the closed loop, after the snare wire has passed through the closed loop. In other embodiments, the capture mechanism is a pair of jaws capable of transitioning from an open to a closed position, the jaws cooperatively forming a capture orifice when in a closed position. The jaws are capable of capturing the snare wire by closing after the snare wire has passed between the jaws. In certain embodiments, the snare wire includes a distal bulb, preventing the wire from slipping back through the jaws or closed loop once captured.

In further embodiments, a snare device comprises a head including at least one channel, a snare wire, at least a portion of which resides in the at least one channel, and a closed loop, at least a portion of which resides in said at least one channel. When said snare wire is advanced, said snare wire extends from said at least one channel along an arcuate path curving back toward said capture mechanism. After said snare wire is advanced at least to said capture mechanism, activation of said capture mechanism captures said snare wire, creating a formed loop. Refraction of at least one of said snare wire and said capture mechanism contracts said formed loop.

This summary is provided to introduce a selection of the concepts that are described in further detail in the detailed description and drawings contained herein. This summary is not intended to identify any primary or essential features of the claimed subject matter. Some or all of the described features may be present in the corresponding independent or dependent claims, but should not be construed to be a limitation unless expressly recited in a particular claim. Each embodiment described herein is not necessarily intended to address every object described herein, and each embodiment does not necessarily include each feature described. Other forms, embodiments, objects, advantages, benefits, features, and aspects of the present invention will become apparent to one of skill in the art from the detailed description and drawings contained herein. Moreover, the various apparatuses and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF DRAWINGS

A better understanding of the present invention will be had upon reference to the following description in conjunction with the accompanying drawings. FIGS. 2-7 depict the stepwise progression of advancement, capture, and retraction of a first embodiment of the snare device. FIG. 8 depicts an intermediate step between FIGS. 2 and 3 at a different view to display additional components of the first embodiment. FIGS. 9-12 depict the stepwise progression of advancement, capture, and retraction of a second embodiment of the snare device. FIGS. 13-16 depict a third embodiment of the snare device. FIGS. 17-24 depict a fourth embodiment of the snare device, with FIGS. 17-21 focusing on the head. The drawings are described in greater detail as follows:

FIG. 1 depicts a perspective view of an open loop snare device.

FIG. 2 depicts a side view of the distal portion of a first embodiment of an open loop snare device with a capture mechanism partially exposed;

FIG. 3 depicts a side view of the distal portion of the first embodiment with the capture mechanism further exposed and having two jaws in an open configuration;

FIG. 4 depicts a side view of the distal portion of the first embodiment with a snare wire deployed along an arcuate path and curling back toward the capture mechanism;

FIG. 5 depicts a side view of the distal portion of the first embodiment with the snare wire deployed along an arcuate path and curling back toward and through the open jaws of the capture mechanism;

FIG. 6 depicts a side view of the distal portion of the first embodiment with the snare wire deployed along an arcuate path captured by the closed jaws of the capture mechanism, with the capture mechanism being partially retracted into the endoscopic snare device;

FIG. 7 depicts a side view of the distal portion of the first embodiment with the snare wire being retracted into the snare device;

FIG. 8 depicts a perspective view of the distal portion of the first embodiment with the snare wire capture mechanism partially exposed;

FIG. 9 depicts a perspective view of the distal portion of a second embodiment of an open loop snare device;

FIG. 10 depicts a perspective view of the distal portion of the second embodiment with a capture mechanism extended, and with a snare wire deployed along an arcuate path and curling back toward the snare wire capture mechanism;

FIG. 11 depicts a perspective view of the distal portion of the second embodiment with the snare wire deployed along an arcuate path and curling back toward and through the closed loop of the capture mechanism;

FIG. 12 depicts a perspective view of the distal portion of the second embodiment with the snare wire deployed along an arcuate path and captured by the retraction of the capture mechanism into the endoscopic snare device.

FIG. 13 depicts a side view of the distal portion of a third embodiment of an open loop snare device, with a snare wire deployed along an arcuate path and curling back toward and through the closed loop of a snare wire capture mechanism.

FIG. 14 depicts a top perspective view of the distal portion of the third embodiment.

FIG. 15 depicts a bottom perspective view of the distal portion of the third embodiment.

FIG. 16 depicts a rear perspective view of the head of the third embodiment of the snare device, with the snare wire and legs of the closed loop shown in cross section along lines 16-16 of FIG. 13.

FIG. 17 depicts a top view of the head of a fourth embodiment of an open loop snare device.

FIG. 18 depicts a side view of the head of the fourth embodiment.

FIG. 19 depicts a bottom perspective view of the head of the fourth embodiment.

FIG. 21 depicts a rear perspective view of the head of the fourth embodiment of the snare device, with the sheath omitted for clarity.

FIG. 22 depicts a side view of the distal portion of the fourth embodiment, with a snare wire deployed along an arcuate path and captured by a refracted closed loop capture mechanism.

FIG. 23 depicts a top view of the distal portion of the fourth embodiment.

FIG. 24 depicts a side view of the distal portion of the fourth embodiment, with the sheath shown in cross section along lines 24-24 of FIG. 23.

FIG. 25(A-D) is a series perspective views sequentially showing steps of a method of using an open loop snare device.

### DETAILED DESCRIPTION OF THE ILLUSTRATED EMBODIMENTS

For the purposes of promoting an understanding of the principles of the invention, reference will now be made to selected embodiments illustrated in the drawings and specific language will be used to describe the same. It will nevertheless be understood that no limitation of the scope of the invention is thereby intended; any alterations and further modifications of the described or illustrated embodiments, and any further applications of the principles of the invention as illustrated herein are contemplated as would normally occur to one skilled in the art to which the invention relates. At least one embodiment of the invention is shown in great detail, although it will be apparent to those skilled in the relevant art that some features or some combinations of features may not be shown for the sake of clarity.

Any reference to “invention” within this document herein is a reference to an embodiment of a family of inventions, with no single embodiment including features that are necessarily included in all embodiments, unless otherwise stated. Further, although there may be references to “advantages” provided by some embodiments of the present invention, it is understood that other embodiments may not include those same advantages, or may include different advantages. Any advantages described herein are not to be construed as limiting to any of the claims.

Specific quantities (spatial dimensions, pressures, momenta, dimensionless parameters, etc.) may be used explicitly or implicitly herein, such specific quantities are presented as examples and are approximate values unless otherwise indicated. Discussions pertaining to specific compositions of matter are presented as examples and do not limit the applicability of other compositions of matter, especially other compositions of matter with similar properties, unless otherwise indicated.

Although primarily discussed as an endoscopic device for performing polypectomies, the open loop snare device disclosed herein may also be adapted for use in surgical procedures, such as, for example, laparoscopy, and may be used to resect tissues of any type.

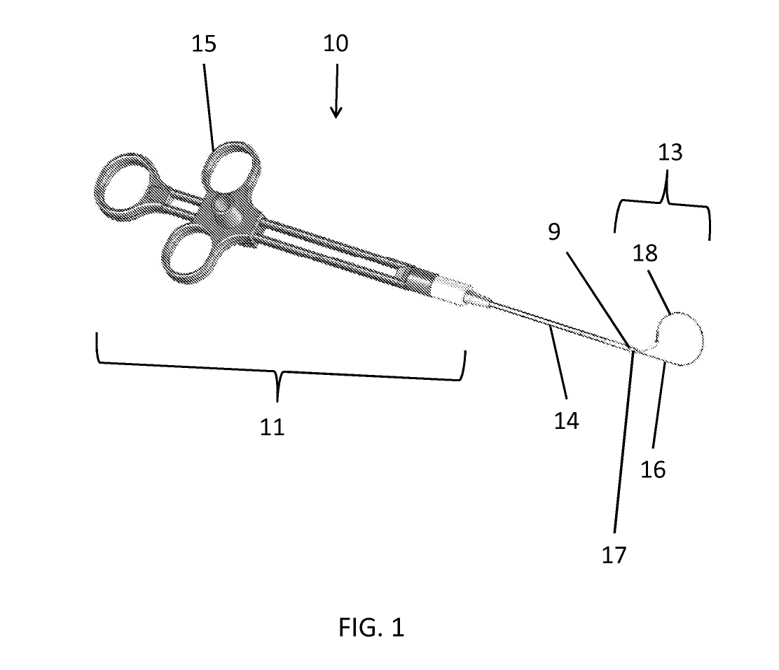

Referring to FIG. 1, a snare device 10 includes a proximal portion 11 and a distal portion 13. The snare device 10 is designed to ensnare polyps or other tissues in proximity to the distal portion 13 of the snare device 10 and to be controlled by a user manipulating the proximal portion 11. The proximal portion 11 is designed to remain outside a patient and includes a means for controlling the distal portion 13, such as, for example, a handle 15. Attached to the handle 15 is an elongated flexible sheath 14. The distal portion 13 of the snare device 10 includes the distal end 9 of the sheath 14, a head 17 contacting the distal end 7 of the sheath 14, and a snare wire 16 and a capture mechanism 18 extending from the head 17. The distal portion 13 is designed to be inserted into a patient.

The snare device 10 is adapted to be channeled to a desired position within a patient by passing the elongated flexible sheath 14 though a channel in an elongated flexible tube adapted for introduction into a patient, such as an endoscopic or laparoscopic tube containing an instrument insertion channel. The sheath 14 preferably has a smaller diameter than that of the instrument insertion channel through which the distal portion 13 is to be channeled such that the distal portion 13 may pass with relative ease through the length of an endoscopic tube, and exit through the distal end of the endoscopic or laparoscopic tube when inserted within a body cavity.

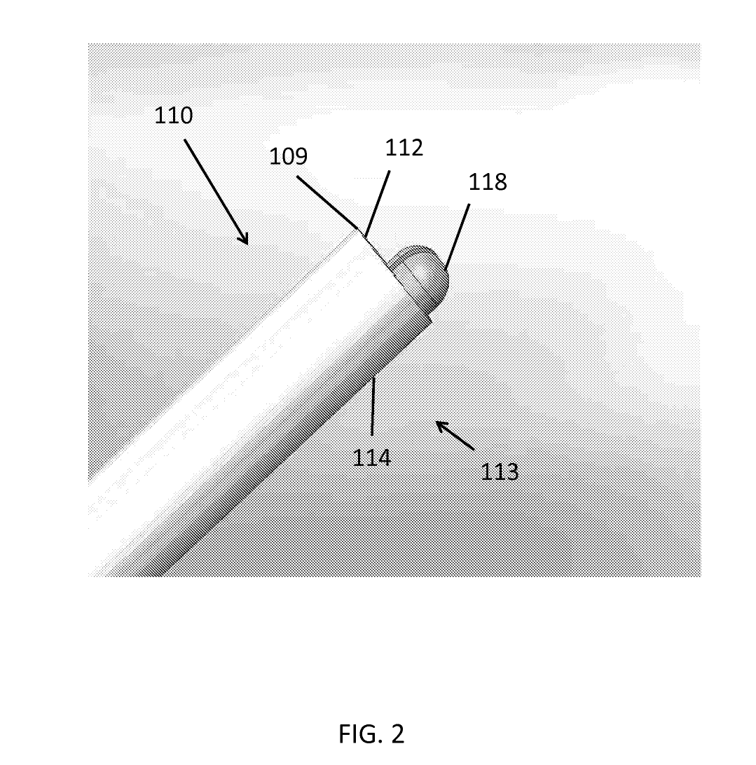

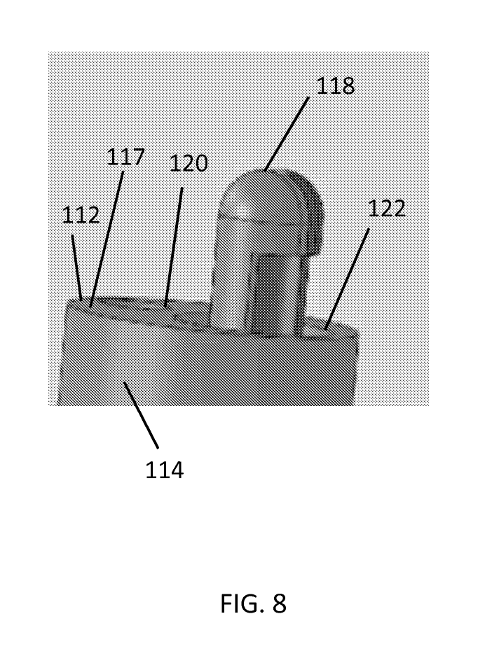

Several embodiments of a snare device are described in detail herein. The distal portion 113 of a first embodiment of an open loop snare device 110 is shown in FIGS. 2-8. In this embodiment, the sheath 114 terminates at a sheath distal end 109. The substantially cylindrical head 117 is inserted within the sheath 114 and terminates at a flat distal end 112 flush with the sheath distal end 109. The head 117 includes at least one channel in which at least a portion of a snare wire 116 and at least a portion of a capture mechanism 118 reside. In some embodiments, the head 117 may include at least two channels, at least one retaining the capture mechanism 118 and at least one retaining the snare wire 116. In embodiments incorporating a plurality of snare wires 116, the snare device 110 may include three or more channels, at least one for the capture mechanism 118 and at least one for each of the plurality of snare wires 116. As shown in FIG. 8, the distal end 112 of the first embodiment of the snare device 110 includes two channels: a snare wire channel 120 and a capture mechanism channel 122.

The capture mechanism 118 is configured to capture the snare wire 116. In the first embodiment depicted in FIGS. 2-8, the capture mechanism 118 is a pair of opposing jaws 124 that can be controllably opened and closed from the proximal end of the open loop endoscopic snare device 110. Activation of the capture mechanism 118, that is, transitioning the jaws 124 from an open position to a closed position, captures the snare wire 116 when the snare wire 116 extends between the jaws 124.

The distal portion 113 can be rotated within the endoscope's instrument insertion channel by manipulating the proximal portion (not shown in this embodiment) of the endoscope to modify the position and orientation of the snare wire 116 and capture mechanism 118 relative to the target polyp or tissue. In certain embodiments, the snare wire channel 120 is preferably slotted or otherwise non-circular, so that the snare wire 116 is maintained at a specific orientation and reliably extends from the snare wire channel 120 on a predetermined plane. In this first embodiment, as shown in FIG. 8, the snare wire channel 120 is substantially circular.

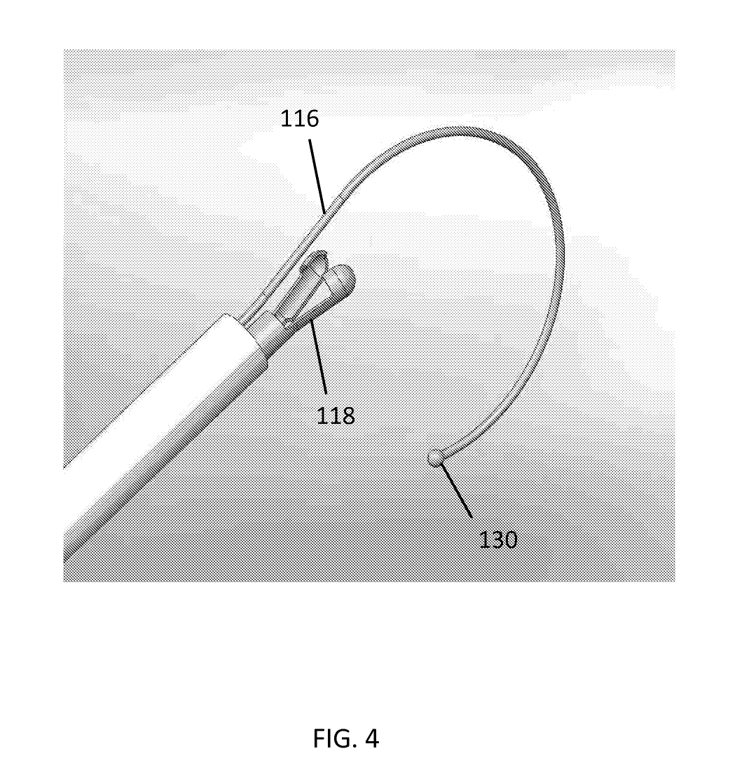

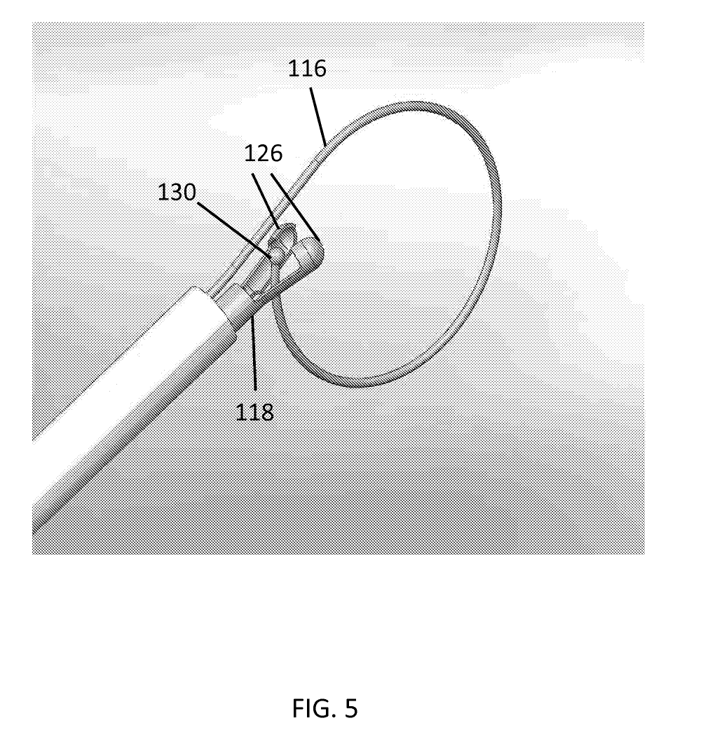

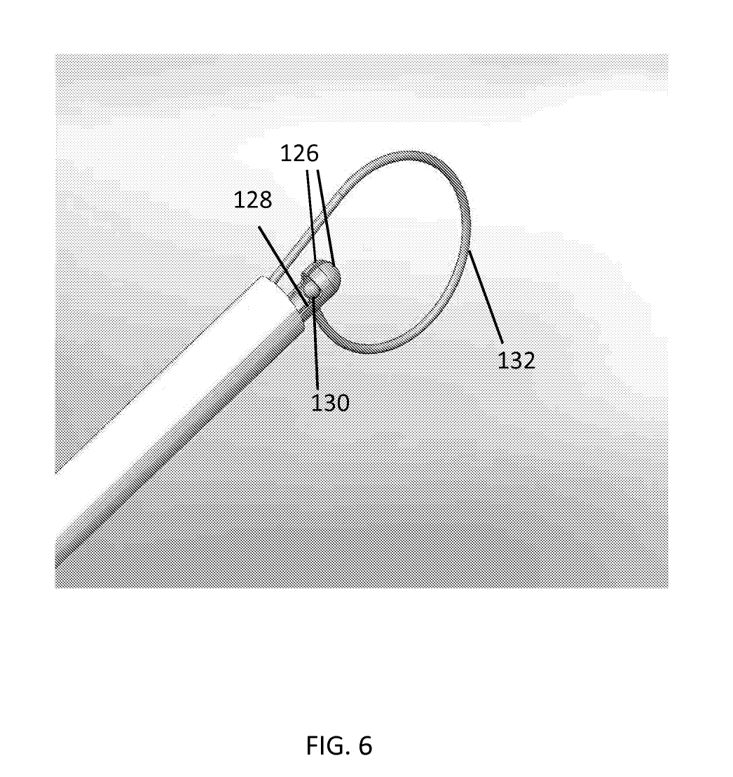

The snare wire 116 is made from a memory shape material such as a memory shape alloy, for example, nickel titanium, also known as nitinol, or a memory shape polymer. As it exits the snare wire channel 120, as shown by FIGS. 4-5, the snare wire 116 is fashioned so as to preferably curl toward the capture mechanism 118 along a predetermined arcuate path to produce a snare of sufficient diameter to ensnare a target polyp or other tissue. The snare wire 116, being made from a memory shape material and being configured to curl back upon a predetermined arcuate path of sufficient diameter, will curl around the stalk of the target polyp and will pass through the open jaws 124 of the capture mechanism 118, as shown in FIG. 5, at which point the jaws 124 will transition from an open position to a closed position, as shown in FIG. 6. When in a closed position, jaw distal portions 126 fit flush or interlock, the jaws 124 thereby forming a snare wire capture orifice 128 surrounding the snare wire 116. The diameter of the snare wire capture orifice 128 is larger than the diameter of the snare wire 116. In this first embodiment, the snare wire includes a distal bulb 130 with a diameter larger than that of the snare wire capture orifice 128, such that the distal bulb 130 cannot pass back through the snare wire capture orifice 128 once captured. The snare wire 116 and the capture mechanism 118, when the jaws 124 are in the closed position capturing the distal bulb 130, cooperatively create a formed loop 132. The formed loop 132 is a closed loop formed when the capture mechanism 118 captures the snare wire 116.

In some embodiments, the capture mechanism 118 is maintained at a predetermined orientation to aid capture of the snare wire 116. In other embodiments, the capture mechanism 118 may be capable of rotating freely in the capture mechanism channel 122. For example, in FIG. 8, the capture mechanism 118 is shown rotated approximately 180 degrees compared to its orientation in FIGS. 2-7.

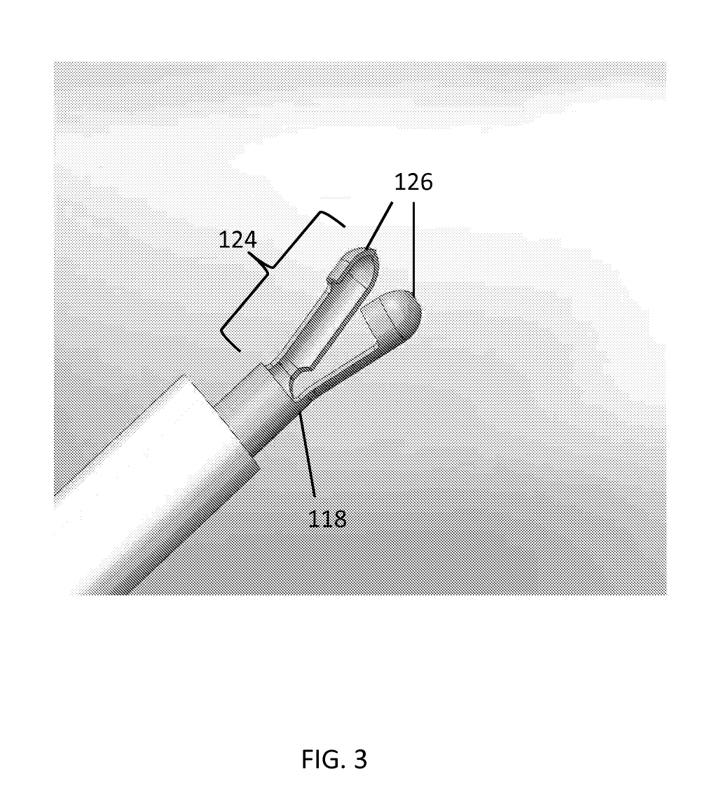

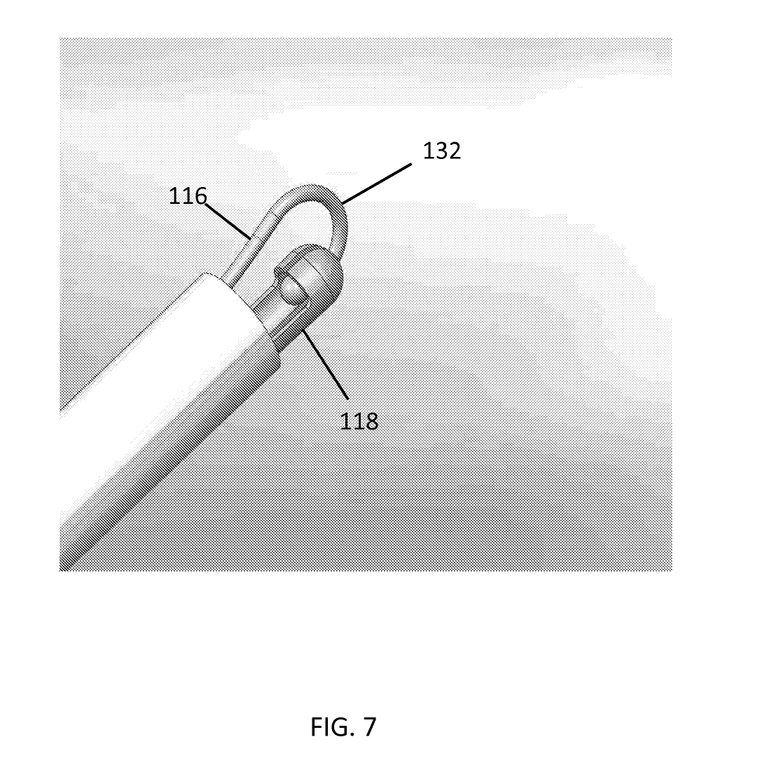

In some embodiments, as shown in FIGS. 3-5, the jaws 124 of capture mechanism 118 are biased in an open position, and may be forced into a closed position by partially retracting them into the capture mechanism channel 122, as shown in FIGS. 6-7, or by advancing an endoscopic tube over the jaws 124. In other embodiments, not shown, the jaws 124 are hingedly attached at their proximal end.

In various embodiments, the diameter of the formed loop 132 may be decreased by at least one of: retracting at least a portion of the capture mechanism 118 into the capture mechanism channel 122 and retracting at least a portion of the snare wire 116 into the snare wire channel 120. In some embodiments, as shown in FIG. 7, both the capture mechanism 118 and snare wire 116 may be retracted into their respective channels 120, 122 to decrease the diameter of the formed loop 132.

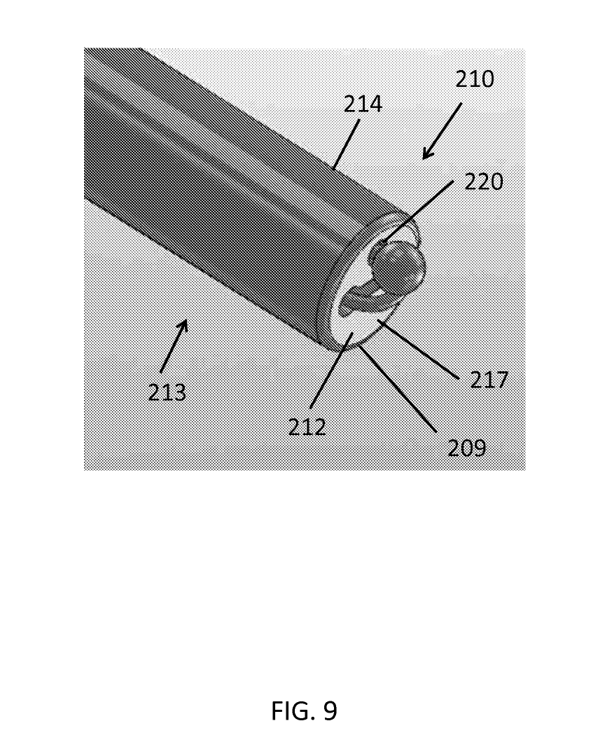

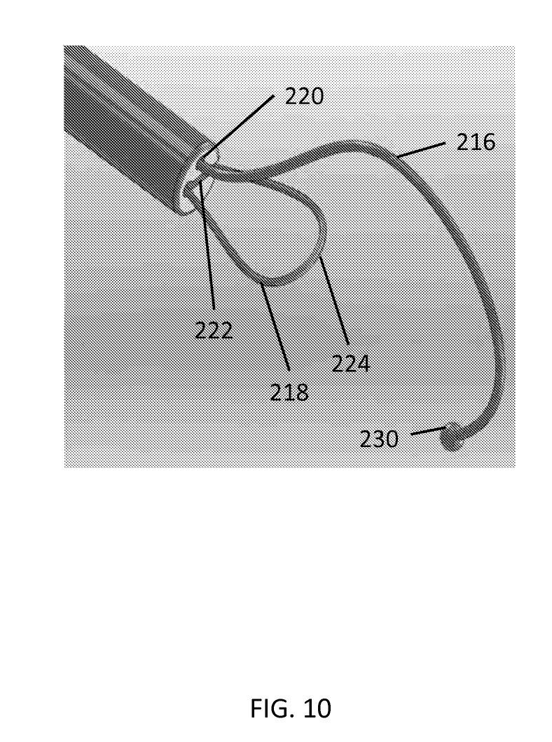

The distal portion 213 of a second embodiment of an open loop snare device 210 is shown in FIGS. 9-12. In this embodiment, the sheath 214 terminates at a sheath distal end 209. The substantially cylindrical head 217 is inserted within the sheath 214 and terminates at a flat distal end 212 flush with the sheath distal end 209. The head 217 includes at least one channel in which at least a portion of a snare wire 216 and a capture mechanism 218 reside. In the second embodiment shown in FIGS. 9-12, the head 217 includes at least two channels: a snare wire channel 220 and a capture mechanism channel 222.

The capture mechanism 218 of the second embodiment is a closed loop 224 that can be controllably extended and retracted from the distal end 212 of the head 217, enlarging and shrinking the diameter of the closed loop 224. The capture mechanism 218 is extended through the snare device 210 via a capture mechanism channel 222. In this second embodiment, as shown in FIGS. 9-12, the capture mechanism channel 222 is generally rectangular or otherwise non-circular in shape, so that the capture mechanism 218 is maintained at a specific orientation and reliably extends from the capture mechanism channel 222 on a predetermined plane. In further embodiments, the capture mechanism channel 222 may be two separate channels, each containing a leg of the capture mechanism 218.

The snare wire 216 is advanced through the head 217 via a snare wire channel 220. The open loop endoscopic snare device 210 can be rotated within an endoscope's instrument insertion channel from the proximal end of the endoscope to modify the orientation of the snare wire 216 and capture mechanism 218 relative to a target polyp or tissue. In certain embodiments, the snare wire channel 220 is preferably slotted or otherwise non-circular, so that the snare wire 216 is maintained at a specific, predetermined orientation and reliably extends from the snare wire channel 220 on a predetermined plane.

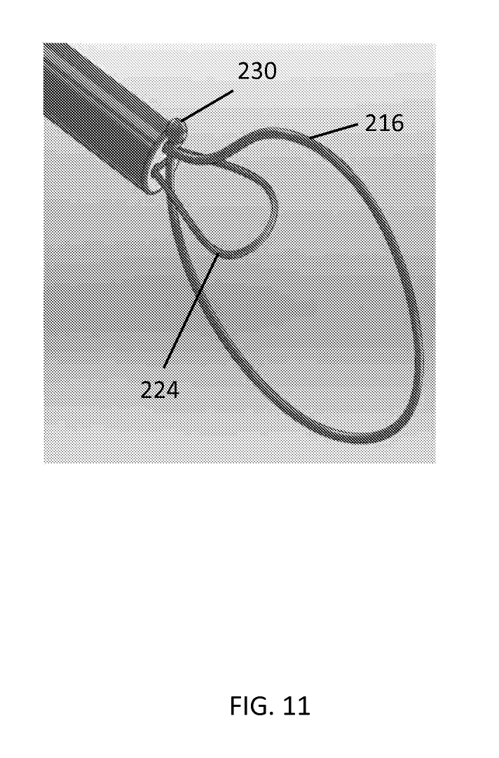

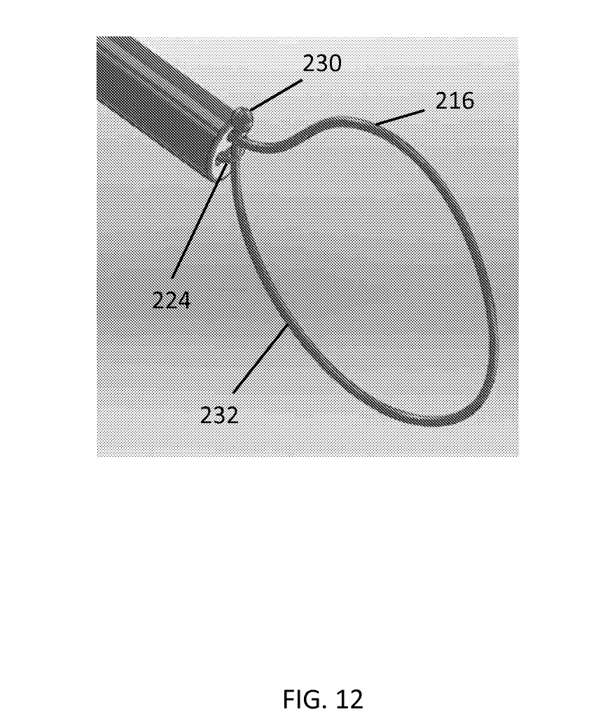

The snare wire 216 is made from a memory shape material such as a memory shape alloy, for example, nickel titanium, or a memory shape polymer. As it exits the snare wire channel 220, as shown by FIGS. 9-12, the snare wire 216 is fashioned so as to curl toward the capture mechanism 218 along a predetermined arcuate path to produce a snare of sufficient diameter to ensnare a target polyp or tissue. The snare wire 216, being made from a memory shape material and being configured to curl back upon a predetermined arcuate path designed to curl around a target tissue and will pass through the extended closed loop 224, as shown in FIGS. 11. As shown in FIG. 12, the capture mechanism 218 can be activated by retracting at least a portion of the closed loop 224 into the capture mechanism channel 222, capturing the snare wire 216 against the head 217 and creating a formed loop 232. The snare wire 216 includes a distal bulb 230 to prevent the wire 216 from reversing back through the capture mechanism 218, once captured. In some embodiments, the distal end 212 includes a recessed portion shaped to receive the distal bulb 130.

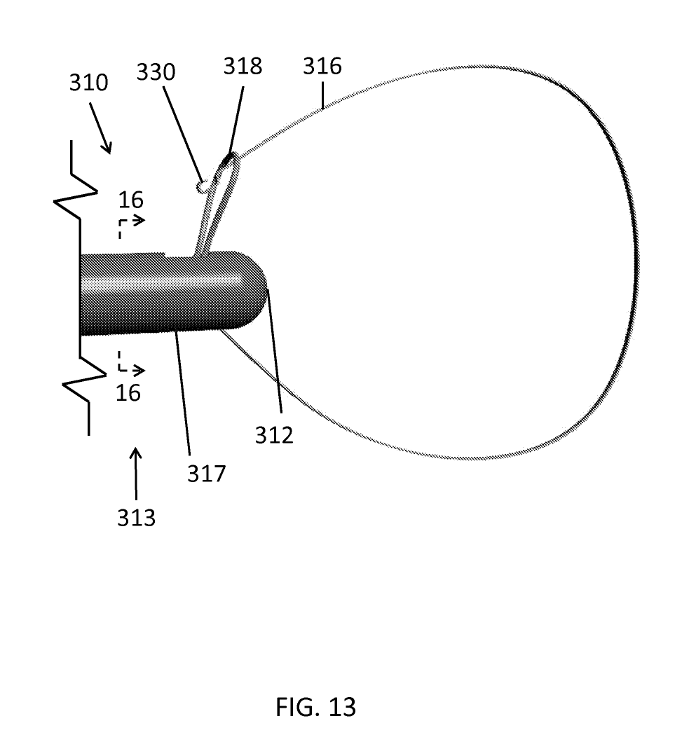

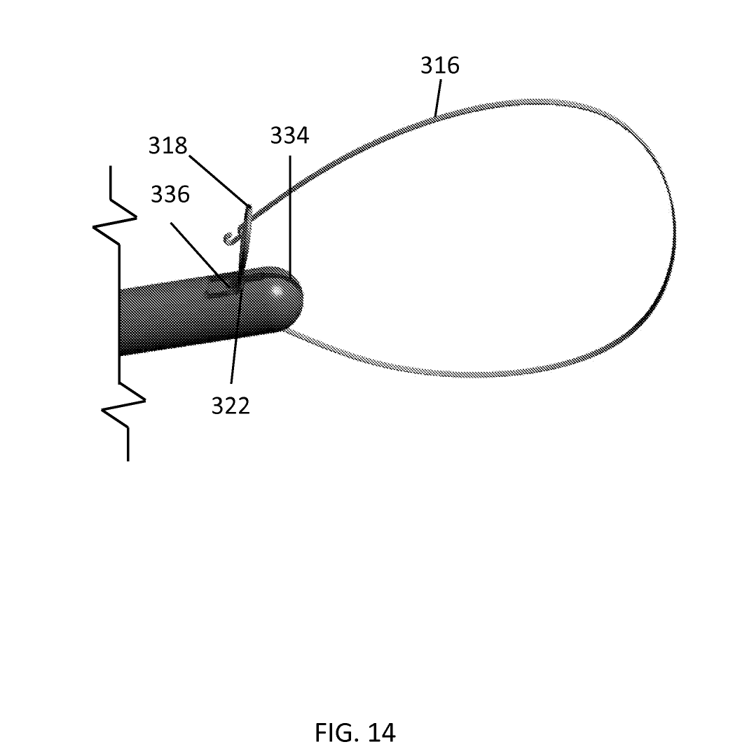

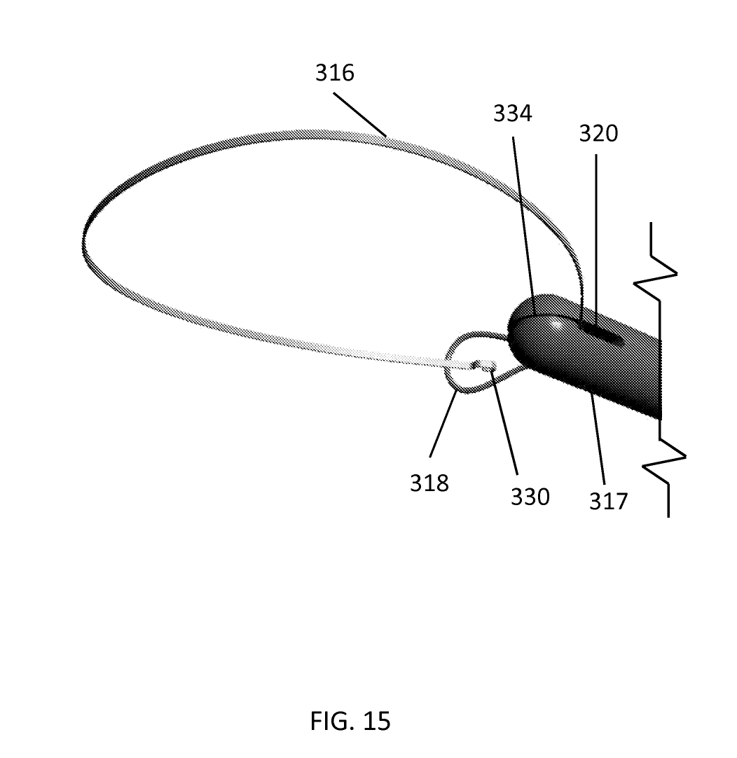

The distal portion 313 of a third embodiment of an open loop snare device 310 is shown in FIGS. 13-16. In this embodiment, the head 317 is generally cylindrical in shape, terminating in a rounded distal end 312. In this third embodiment, the snare device 310 possesses a snare channel 320 located at the bottom of the head 317 and a pair of capture mechanism channels 321, 322 located at the top of the head 317. The snare device 310 may be freely rotated, so the description of channels or other features at the “top” or “bottom” of the head 317 only describes their positions relative to each other in the referenced drawings, and does not limit the orientation of the snare device 310 as a whole.

The capture mechanism 318 of the third embodiment is a closed loop 324 that can be controllably extended and retracted from the distal portion 312 of the snare device 310, enlarging and shrinking the closed loop 324. At least a portion of each leg 323, 325 of the closed loop 324 extends through the head 317 via a separate capture mechanism channel 321, 322. By using two separate capture mechanism channels 321, 322, the closed loop 324 is maintained at a specific orientation and reliably extends from the channels 321, 322 on a predetermined plane. The closed loop 324 may be extended or retracted by extending or retracting either leg 323, 325 of the closed loop 324 individually or both legs 323, 325 of the loop in combination. In some embodiments, one of the legs 323, 325 of the closed loop 324 may be fixed to the head 317 such that only the other, non-fixed leg may be extended or retracted. In this third embodiment, neither leg 323, 325 is fixed to the head 317.

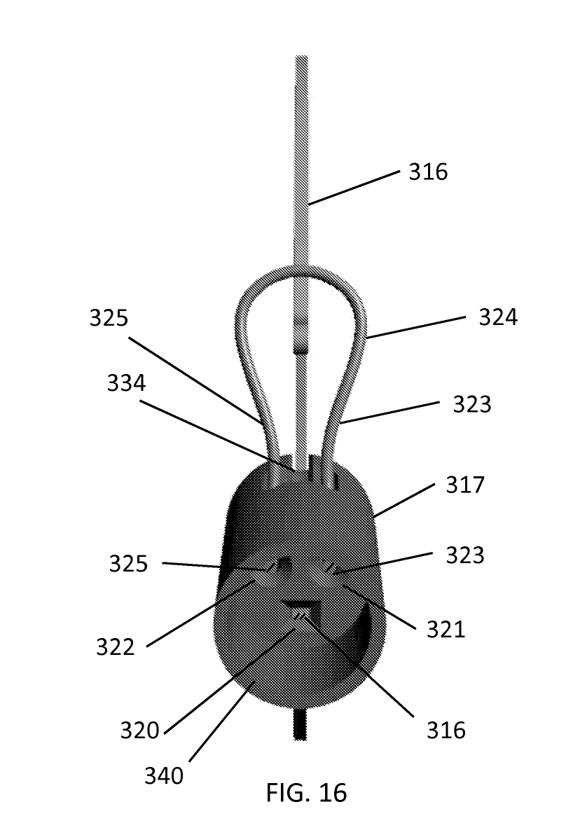

The snare wire 316 is advanced through the snare device 310 via a snare wire channel 320. In this embodiment, as best shown in FIG. 16, the snare wire 316 is a flattened, substantially rectangular shape, at least a portion of which resides in a substantially rectangular snare wire channel 320. The snare wire 316 is unable to rotate within the snare wire channel 320, thus maintaining the snare wire 316 at a predetermined orientation.

In this embodiment, the distal portion 313 includes a slot 334 extending from the snare wire channel 320, over the distal end 312, in the direction of the capture mechanism 318. The slot 334 is sized to accept the snare wire 316 such that the snare wire 316, when refracted after being captured by the capture mechanism 318, enters into the slot 334 and is thereby maintained at a specific orientation.

The snare wire 316 is made from a memory shape material such as a memory shape alloy, for example, nickel titanium, or a memory shape polymer. As it exits the snare wire channel 320, as shown by FIGS. 13-16, the snare wire 316 is fashioned so as to curl toward the capture mechanism 318 along a predetermined arcuate path to produce a snare of sufficient diameter to ensnare a target polyp or tissue. The snare wire 316, being made from a memory shape material and being configured to curl back upon a predetermined arcuate path of sufficient diameter, will curl around the stalk of the target polyp and will pass through the extended capture mechanism 318. The capture mechanism 318 can then be activated by retracting at least a portion of the closed loop 324 into at least one of the capture mechanism channels 321, 322, decreasing the diameter of the closed loop 324 and capturing the snare wire 316 against the head 317, thereby creating a formed loop (not shown for this embodiment). The snare wire 316 includes a distal hook 330 to catch the closed loop 324 and prevent the wire 316 from reversing back through the capture mechanism 318, once captured. In this embodiment, the head 317 includes a recessed portion 336 shaped to receive the distal hook 330, once captured.

The head 317 is adapted to contact the distal end of the sheath (not shown). Unlike the head 117, 217 in the first two embodiments, the head 317 in the third embodiment is shaped to fit over the distal end of the sheath. As shown in FIG. 16, the head 317 includes a cavity 340 sized to accept the distal end of the sheath. In various embodiments, the head may be adapted for at least a portion of the head to be inserted into the distal end of the sheath, for the distal end of the sheath to be inserted into the head, to abut the distal end of the sheath, or otherwise contact and be secured to the distal end of the sheath.

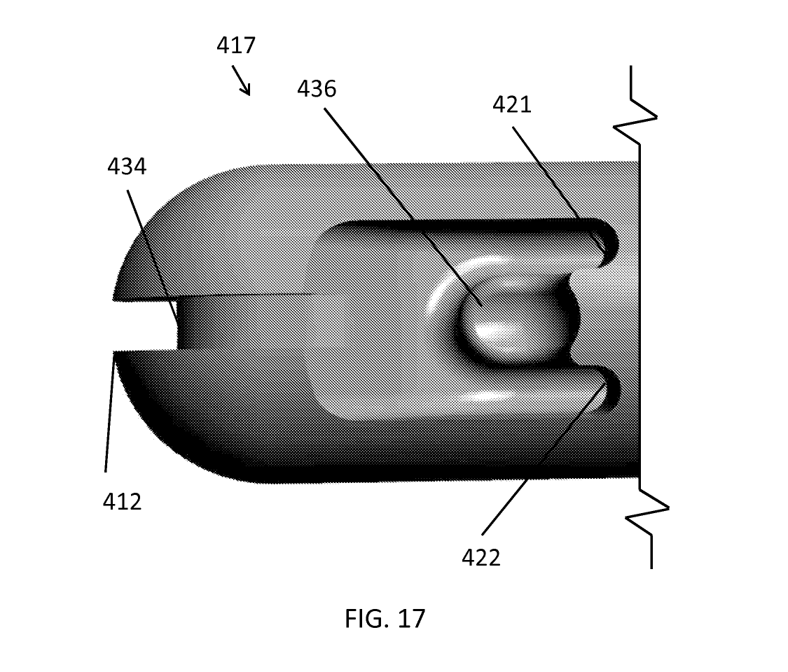

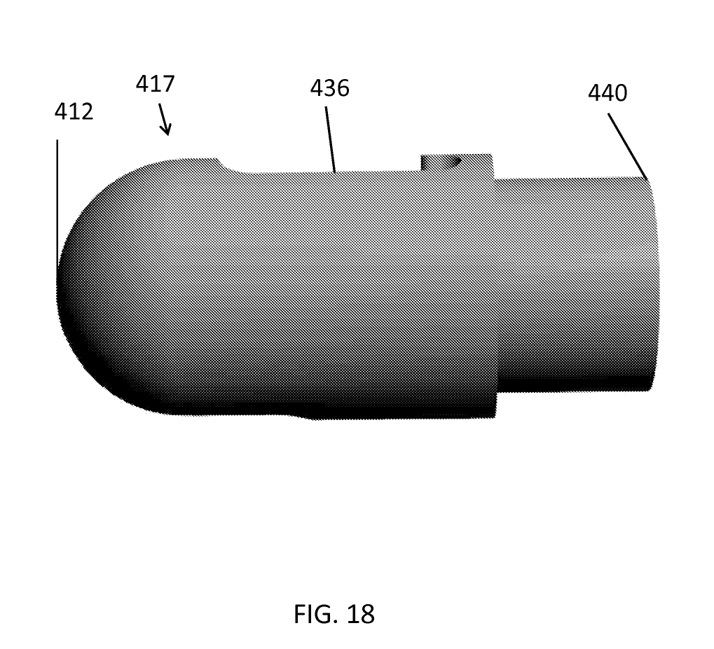

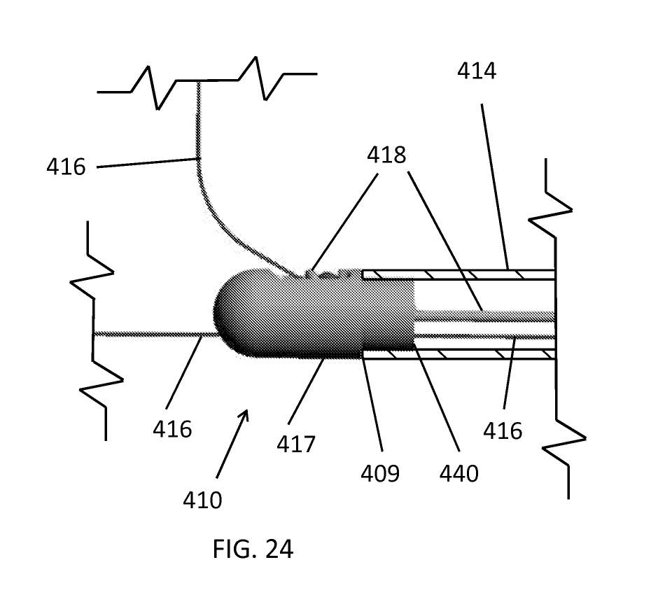

The distal portion 413 of a fourth embodiment of an open loop snare device 410 is shown in FIGS. 17-24, with FIGS. 17-20 focusing on the head 417. In this embodiment, the head 417 is generally cylindrical in shape, terminating in a rounded distal end 412 and having a decreased diameter at its proximal end 440 to aid insertion of the proximal end 440 of the head 417 into the sheath 414, as shown in FIG. 24, contacting the sheath distal end 409. In this fourth embodiment, the snare device 410 possesses a snare wire channel 420 located at the bottom of the head 417 and a pair of capture mechanism channels 421, 422 located at the top of the distal portion 413, within a recessed portion 436. The snare device 410 may be freely rotated, so the description of channels or other features at the “top” or “bottom” of the head 417 only describes their positions relative to each other in the referenced drawings, and does not limit the orientation of the snare device 410 as a whole.

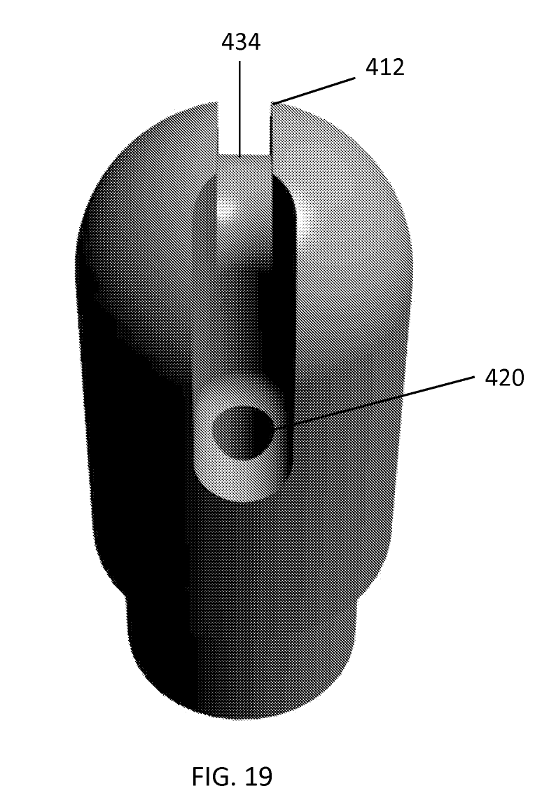

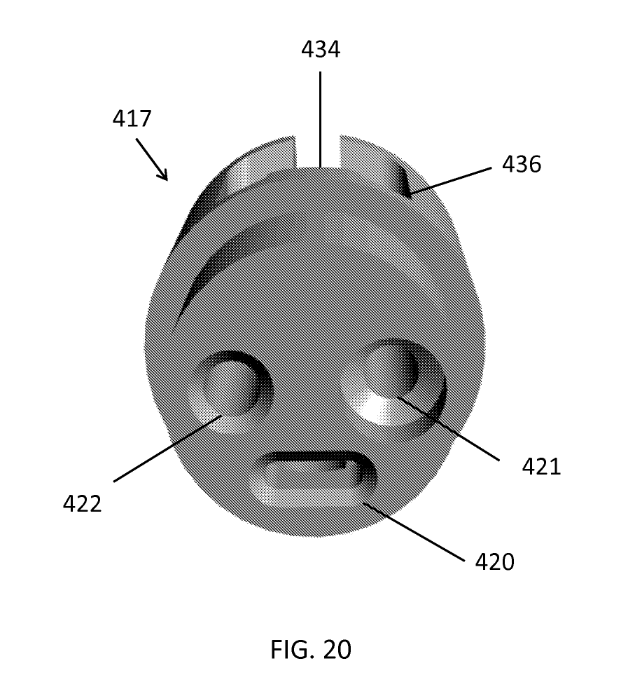

As shown in FIGS. 19 and 20, the snare wire channel 420 has a round exit but the interior of the channel 420 is substantially rectangular in shape. In this fourth embodiment, the substantially rectangular interior of the snare wire channel 420 maintains the snare wire 416 at a specific orientation, as the wire 416 cannot rotate within the channel 420.

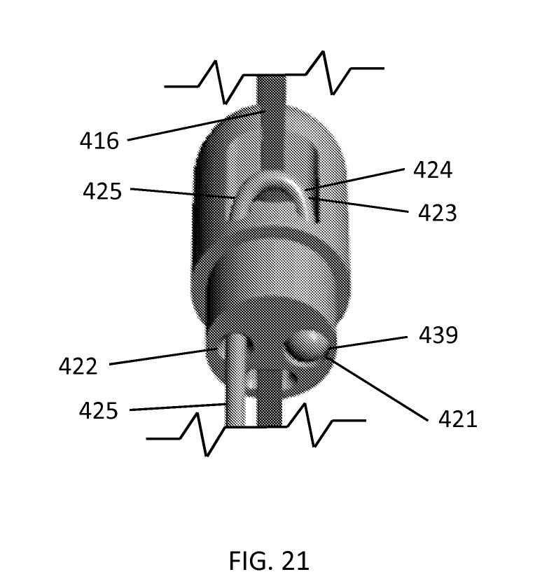

The capture mechanism 418 of the fourth embodiment is a closed loop 424 that can be controllably extended and retracted from the distal portion 412 of the snare device 410, enlarging and shrinking the closed loop 424. Each leg 423, 425 of the closed loop 424 is extended through the snare device 410 via a separate capture mechanism channel 421, 422. By using two separate capture mechanism channels 421, 422, the closed loop 424 is maintained at a specific orientation and reliably extends from the channels 421, 422 on a predetermined plane. As shown in FIG. 21, in this fourth embodiment, leg 423 of the closed loop 424 is fixed to the head 417 such that only the other, non-fixed leg 425 may be extended or retracted. In this fourth embodiment, the fixed leg 423 terminates in a bulb 439 with a diameter greater than the diameter of the capture mechanism channel 421. The capture mechanism 418 is thereby formed by single wire extending from the bulb 439, through the capture mechanism channel 421, out of the head 417 to form the closed loop 424, returning through the capture mechanism channel 422, and extending proximally down the sheath 414. In other embodiments, other means for fixing a leg to the head may be used.

The snare wire 416 is advanced through the snare device 410 via a snare wire channel 420. In this embodiment, the distal portion 413 includes a slot 434 extending from the snare wire channel 420, over the distal end 412, in the direction of the capture mechanism 418. The slot 434 is sized to accept the snare wire 416 such that the snare wire 416, when retracted after being captured by the capture mechanism 418, enters into the slot 434 and is thereby maintained at a specific orientation.

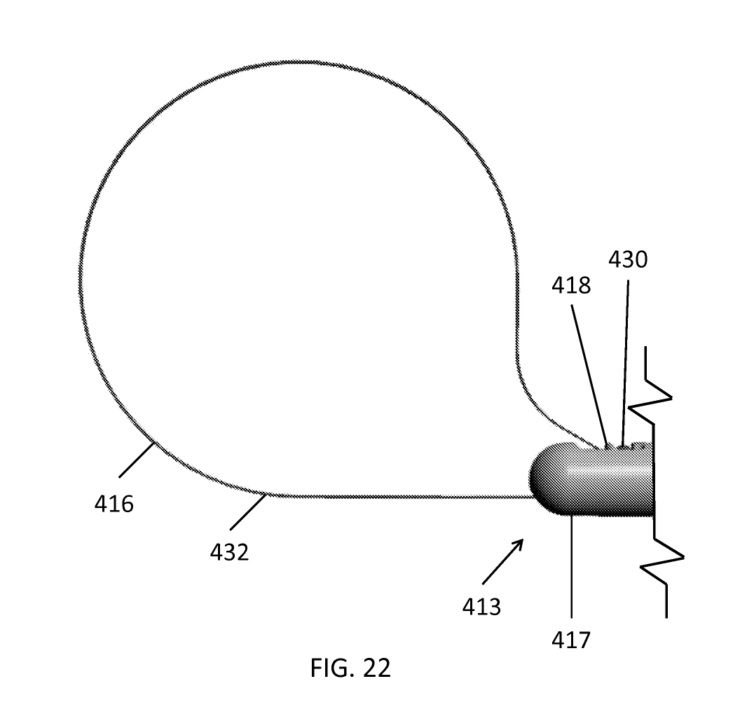

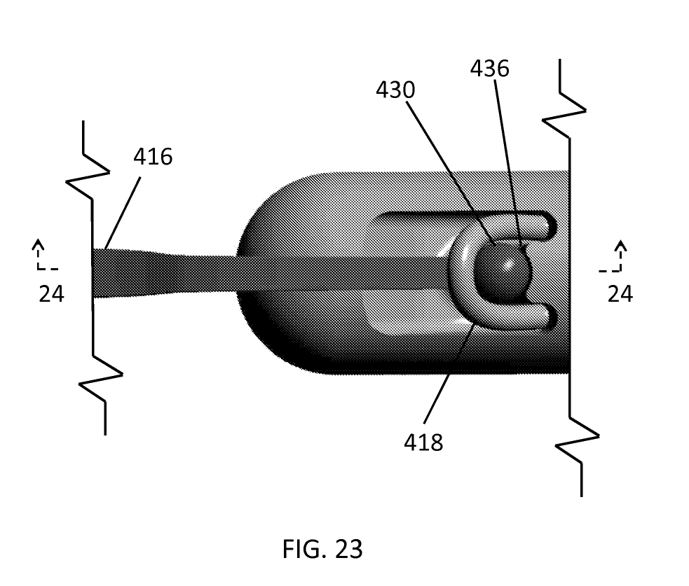

The snare wire 416 is made from a memory shape material such as a memory shape alloy, for example, nickel titanium, or a memory shape polymer. As in other embodiments, the snare wire 416 is fashioned so as to curl toward the capture mechanism 418 along a predetermined arcuate path to produce a snare of sufficient diameter to ensnare a target polyp or tissue and pass through the extended capture mechanism 418. The capture mechanism 418 can then be retracted into the capture mechanism channels 421, 422, capturing the snare wire 416 and creating a formed loop 432. The snare wire 416 includes a distal bulb 430 to prevent the wire 416 from reversing back through the capture mechanism 418, once captured. In this embodiment, the distal portion 412 includes a recessed portion 436 shaped to receive the distal bulb 430. As shown in FIGS. 22 and 23, once the snare wire 416 is captured, retracting the capture mechanism 418 secures the distal bulb 430 in the recessed portion 436.

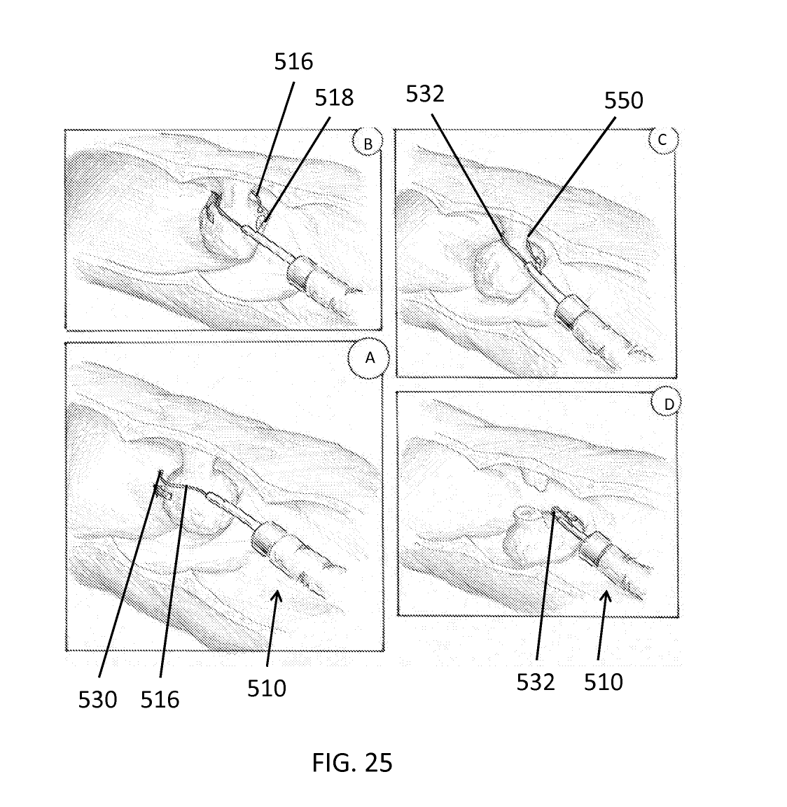

Referring now FIG. 25(A-D), when the disclosed open loop snare device 510 is used in a polypectomy procedure, the snare wire 516 may be extended to surround a polyp stalk 550 (panels A-B), then the distal bulb 530 be captured by the capture mechanism 518, forming the formed loop 532 around the polyp stalk 550 (panel C). The polyp stalk 550 can be incised across the width of the stalk by decreasing the diameter of the formed loop 532, or by retraction of the snare device 510 of the along the path of the endoscope as the endoscope is withdrawn from the colon, or as the snare device 510 is refracted into the instrument insertion channel of the endoscope (panel D).

Optionally, the formed loop 132, 232, 332, 432 created by the snare wire 116, 216, 316, 416 and the capture mechanism 118, 218, 318, 418 completes a circuit across which current may be applied. This circuit will allow for the delivery of a bipolar current, which provides an integrated path for current return from the body to the current generator. This configuration may avoid the need for a grounding pad for bleeding current from the body, as is used with snares with a monopolar current configuration. The current may range from a level of amperes known by those skilled in the art to facilitate cutting up to a level of amperes known to those skilled in the art to facilitate coagulation and sealing of the wound created by the polyp removal. The known resistance of the snare wire facilitates the accurate calculation of the heat generated by electrically charging the snare wire. In one embodiment, the formed loop 132, 232, 332, 432 is a means for biopolar current delivery in which electrical current travels from the snare wire 116, 216, 316, 416 to the capture mechanism 118, 218, 318, 418.

After incision of a polyp stalk, the bleeding polyp stalk remnant is preferably sealed to prevent bleeding. In one embodiment, the stalk remnant can be cauterized by the application of sufficient electric current through the snare wire 116, 216, 316, 416. In a further embodiment, the stalk remnant is sealed by application of a ligature. In a still further embodiment the stalk remnant is sealed both by application of a ligature and by cauterization by the charged snare wire 116, 216, 316, 416.

In certain embodiments, the capture mechanism and snare wire are controlled by control wires (not shown) affixed to the capture mechanism and snare wire, passing through the respective snare wire and capture mechanism channels, and extending to the proximal portion of the snare device, where the control wires may be manipulated by the user. For example, advancing the control wire attached to the snare wire would extend the snare wire, and withdrawing the same control wire would retract the snare wire into the snare wire channel.

Further embodiments (not shown) incorporate a first magnet on the distal end of the snare wire and a second a magnet or an electromagnet on the capture mechanism or the head to facilitate the capture of the snare wire, or alternatively, to guide the snare wire to the capture mechanism. In these embodiments, the first magnet may act as a complement to or a replacement for the distal bulb or distal hook. In these embodiments, the second magnet may be positioned to attract and secure the first magnet, when the snare wire has been captured.

The snare device disclosed herein is adapted to be channeled to a desired position within a patient by passing the snare device though a channel in an elongated flexible tube adapted for introduction into a patient, such as an endoscopic tube containing an instrument insertion channel. In some embodiments, the snare device may be used in conjunction with at least one additional endoscopic device. For example, an endoscopic tube may include two instrument insertion channels, one for the snare device disclosed herein and the other for an endoscopic camera. In some embodiments, the camera may be used to detect cancerous masses or other target tissue and the snare device used to remove the identified masses during a single colonoscopy procedure. By viewing the environment local to the distal end of the snare device using the camera, the user may watch the progress of the snare wire and manipulate the snare device so as to capture the snare wire using the capture mechanism when the snare wire is in the desired position.

Various aspects of different embodiments of the present disclosure are expressed in paragraphs X1 and X2 as follows:

X1. One embodiment of the present disclosure includes a snare device comprising: a snare wire; and a capture mechanism; wherein when the snare wire is advanced, the snare wire extends from a distal portion of the device along an arcuate path curving back toward the capture mechanism; wherein after the snare wire is advanced at least to the capture mechanism, activation of the capture mechanism captures the snare wire, creating a formed loop; and wherein retraction of at least one of the snare wire and capture mechanism contracts the formed loop.

X2. Another embodiment of the present disclosure includes a snare device comprising: a head including at least one channel; a snare wire, at least a portion of the snare wire residing in the at least one channel; and a closed loop, at least a portion of the closed loop residing in the at least one channel; wherein when the snare wire is advanced, the snare wire extends from the at least one channel along an arcuate path curving back toward the closed loop;

wherein, after the snare wire is advanced through the closed loop, retraction of the closed loop the captures the snare wire, creating a formed loop; and wherein retraction of at least one of the snare wire and the capture mechanism contracts the formed loop.

Yet other embodiments include the features described in any of the previous paragraphs X1 or X2, as combined with one of more of the following aspects:

Wherein the capture mechanism includes opposing jaws.

Wherein the capture mechanism includes a closed loop.

Wherein the head includes a recessed portion.

Wherein the snare wire includes one of a distal bulb and a distal hook.

Wherein the head includes a recessed portion shaped to receive one of a distal bulb and a distal hook.

Wherein activation of the capture mechanism comprises transitioning opposing jaws from an open position to a closed position.

Wherein activation of the capture mechanism comprises retraction of the closed loop.

Wherein the snare device includes a head contacting a distal end of a sheath.

Wherein the snare device includes a head at least partially inserting within the distal end of a sheath.

Wherein the head includes at least one snare wire channel and at least one capture mechanism channel.

Wherein the head includes one snare wire channel and one capture mechanism channel.

Wherein the head includes one snare wire channel and two capture mechanism channels.

Wherein the head includes a notch sized to receive said snare wire.

Wherein creation of said formed loop completes a circuit for delivery of electrical current.

Reference systems, if used herein, refer generally to various directions (for example, top, bottom, upper, lower, forward, rearward, left, right, etc.), which are merely offered to assist the reader in understanding the various embodiments of the disclosure and are not to be interpreted as limiting. Other reference systems may be used to describe various embodiments.

While illustrated examples, representative embodiments and specific forms of the invention have been illustrated and described in detail in the drawings and foregoing description, the same is to be considered as illustrative and not restrictive or limiting. The description of particular features in one embodiment does not imply that those particular features are necessarily limited to that one embodiment. Features of one embodiment may be used in combination with features of other embodiments as would be understood by one of ordinary skill in the art, whether or not explicitly described as such. Exemplary embodiments have been shown and described, and all changes and modifications that come within the spirit of the invention are desired to be protected.

What is claimed is:

1) A snare device comprising:
a snare wire; and
a capture mechanism;
wherein, when the snare wire is advanced, the snare wire extends from a distal portion of the device along an arcuate path curving back toward the capture mechanism;
wherein, after the snare wire is advanced at least to the capture mechanism, activation of the capture mechanism captures the snare wire, creating a formed loop; and
wherein retraction of at least one of the snare wire and capture mechanism contracts the formed loop.

2) The snare device of claim 1, further comprising a head including at least one channel, wherein at least a portion of said snare wire and at least a portion of said capture mechanism reside in said at least one channel.

3) The snare device of claim 2, further comprising an elongated flexible sheath, wherein said head contacts a distal end of said sheath.

4) The snare device of claim 3, wherein said head is at least partially inserted within said distal end of said sheath.

5) The snare device of claim 2, wherein said at least one channel includes at least one snare wire channel and at least one capture mechanism channel, and wherein at least a portion of said snare wire resides in said at least one snare wire channel and at least a portion of said capture mechanism resides in said at least one capture mechanism channel.

6) The snare device of claim 5, wherein said at least one snare wire channel is configured to maintain said snare wire at a predetermined orientation.

7) The snare device of claim 2, wherein said snare wire includes one of a distal bulb and a distal hook.

8) The snare device of claim 7, wherein said head includes a recessed portion shaped to receive one of said distal bulb and said distal hook.

9) The snare device of claim 2, wherein said head includes a notch sized to receive said snare wire.

10) The snare device of claim 1, wherein said capture mechanism includes a pair of opposing jaws capable of transitioning from an open position to a closed position.

11) The snare device of claim 10, wherein said opposing jaws, when in a closed position, cooperative form a snare wire capture orifice.

12) The snare device of claim 11, wherein said snare wire includes a distal bulb having a diameter larger than said snare wire capture orifice.

13) The snare device of claim 11, wherein when said snare wire is advanced, said snare wire extends from a distal portion of the snare device along an arcuate path curving back toward and through the opposing jaws, and wherein transitioning the opposing jaws from an open position to a closed position captures the snare wire, creating a formed loop.

14) The snare device of claim 1, wherein said capture mechanism includes a closed loop.

15) The snare device of claim 14, wherein said closed loop is capable of being extended, increasing its diameter, and retracted, decreasing its diameter.

16) The snare device of claim 15, wherein when said snare wire is advanced, said snare wire extends from a distal portion of the snare device along an arcuate path curving back toward and through said closed loop, and wherein retraction of said closed loop captures said snare wire, creating a formed loop.

17) The snare device of claim 1, wherein said snare wire is formed of a memory shape material.

18) The snare device of claim 1, wherein creation of said formed loop completes a circuit for delivery of electrical current.

19) A snare device comprising:
a head including at least one channel;
a snare wire, at least a portion of said snare wire residing in said at least one channel; and
a closed loop, at least a portion of said closed loop residing in said at least one channel;
wherein when said snare wire is advanced, said snare wire extends from said at least one channel along an arcuate path curving back toward said closed loop;
wherein, after said snare wire is advanced through said closed loop, retraction of said closed loop captures said snare wire, creating a formed loop; and
wherein retraction of at least one of said snare wire and said capture mechanism contracts said formed loop.

20) The snare device of claim 19, further comprising an elongated flexible sheath including a distal end, wherein said head contacts the distal end of said sheath.

21) The snare device of claim 19, wherein said at least one channel includes a snare wire channel and at least one capture mechanism channel, at least a portion of said snare wire residing in said snare wire channel and at least a portion of said closed loop residing in said at least one capture mechanism channel.

22) The snare device of claim 19, wherein said snare wire includes one of a distal bulb and a distal hook.

23) The snare device of claim 22, wherein said head includes a recessed portion shaped to receive one of said distal bulb and said distal hook.
